(19) United States
(12) Patent Application Publication
(10) Pub. No.: US 2018/0272103 A1
(43) Pub. Date: Sep. 27, 2018

(54) INTEGRATE CLIMATE CONTROL WITH RESPIRATORY MONITORING

(71) Applicant: KONINKLIJKE PHILIPS N.V., EINDHOVEN (NL)

(72) Inventors: John E. CRONIN, BONITA SPRINGS, FL (US); Seth Melvin CRONIN, BONITA SPRINGS, FL (US)

(21) Appl. No.: 15/761,473

(22) PCT Filed: Sep. 26, 2016
(86) PCT No.: PCT/EP2016/072780

§ 371 Date: Mar. 20, 2018

Related U.S. Application Data
(60) Provisional application No. 62/232,167, filed on Sep. 24, 2015.

(30) Foreign Application Priority Data
Mar. 18, 2016 (EP) 16161280.9

(51) Int. Cl. A61M 21/02; A61B 5/1455; A61B 5/00; A61G 7/018; F24F 11/88

(52) U.S. Cl. CPC A61M 21/02; A61B 5/14551; A61M 2021/0044; A61G 7/018; F24F 11/88; A61B 5/7425

(57) ABSTRACT

A method for adjusting environmental parameters for patient condition monitoring comprising: inputting via a user interface a patient condition and other information relating to the patient; searching a database for environmental parameters and other information relating to the patient condition; determining environmental parameters to adjust based on the patient condition; defining oxygen saturation level threshold ranges corresponding to various sets of environmental parameters; measuring via a pulse oximeter a first oxygen saturation level of the patient under a first set of environmental parameters; adjusting the environmental parameters corresponding to the patient condition if the first oxygen saturation level lies outside the threshold ranges defined for the first set of environmental parameters; and measuring a second oxygen saturation level of the patient under the adjusted environmental parameters.

### BACKGROUND OF THE INVENTION

Pulse Oximetry is an effective non-invasive method for measuring SpO2. SpO2 level is a good indicator of the patient's respiratory condition. Examples of respiratory diseases that are monitored using pulse oximeters are chronic obstructive pulmonary disease (COPD), asthma, and sleep apnea. Some respiratory diseases, such as the diseases mentioned, are affected by environmental factors such as temperature, humidity, airflow rate, and illumination as well as the physical position of the patient such as bed height and bed angle.

### SUMMARY OF THE CLAIMED INVENTION

Embodiments of the present invention relates to systems and methods for adjusting environmental parameters for patient condition monitoring. The system according to some embodiments comprises a pulse oximeter for measuring an oxygen saturation level of a patient and an environment controller for adjusting the environmental parameters based on the patient condition.

The method according to some embodiments comprises inputting via a user interface a patient condition and other information relating to the patient. The pulse oximeter measures a first oxygen saturation level of the patient under a first set of environmental parameters. Then, the environment controller adjusts the environmental parameters corresponding to the patient condition if the first oxygen saturation level lies outside the threshold ranges corresponding to the first set of environmental parameters. Afterwards, the pulse oximeter measures a second oxygen saturation level of the patient under the adjusted environmental parameters.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a block diagram of a system for suggesting additional tests based on a patient's health status and medical records according to a preferred embodiment of the present invention.

FIG. 2 illustrates a flowchart of a method according to a preferred embodiment of the present invention.

FIG. 3 illustrates a block diagram of a patient monitoring device according to an embodiment of the present invention.

FIGS. 4A and 4B illustrate a user interface according to an embodiment of the present invention.

FIG. 5 illustrates a flowchart of the environment control software.

### DETAILED DESCRIPTION OF THE EMBODIMENTS

Embodiments of the present invention relates to a method for adjusting environmental parameters for patient condition monitoring comprising: inputting via a user interface a patient condition and other information relating to the patient; searching a database for environmental parameters and other information relating to the patient condition; determining environmental parameters to adjust based on the patient condition; defining oxygen saturation level threshold ranges corresponding to various sets of environmental parameters; measuring via a pulse oximeter a first oxygen saturation level of the patient under a first set of environmental parameters; adjusting the environmental parameters corresponding to the patient condition if the first oxygen saturation level lies outside the threshold ranges defined for the first set of environmental parameters; and measuring a second oxygen saturation level of the patient under the adjusted environmental parameters.

Embodiments of the present invention also relates to a system for suggesting additional tests based on a patient's health status and medical records comprising: a pulse oximeter for measuring an oxygen saturation level of a patient; a display device with a user interface for inputting a patient condition and other information relating to the patient; an environment controller for adjusting the environmental parameters; and a patient monitoring device connected to the pulse oximeter, the display device, and the environment controller.

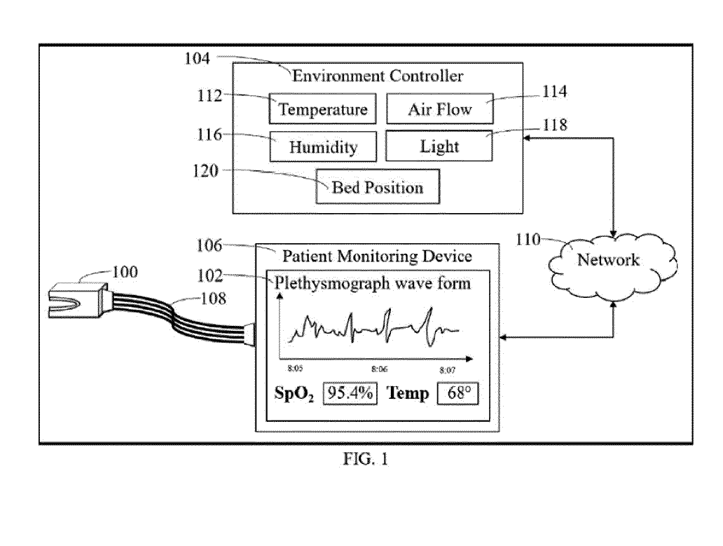

In a preferred embodiment of a system of the present invention as illustrated in FIG. 1, a system for adjusting environmental parameters for patient condition monitoring comprises a pulse oximeter 100, a display device with a user interface 102, an environment controller 104, and a patient monitoring device 106. The pulse oximeter 100 is connected to the patient monitoring device 106 via a wire link 108 in the instant embodiment, although other connection means would also suffice, including wireless communications link such as Wi-Fi, Bluetooth, NFC, infrared, and other means appreciated by those skilled in the art. The patient monitoring device 106 is further connected to the environment controller 104 via a communications link 110. The environment controller 104 is capable of controlling the temperature 112, air flow 114, humidity 116, light 118, and bed position 120 inside a room.

Preferably, the environment controller 104 sends control signals to different devices to adjust environmental parameters. Examples of environmental parameters are temperature 112, humidity 116, air flow/pressure 114, illumination 118, and bed position 120. Examples of devices that control the environmental parameters include HVAC unit, humidifier, LED bulbs, and automatic bed. The transmission of control signals from the environment controller 104 may be accomplished physically via, for examples, a USB or wirelessly via, for example, Wi-Fi communications.

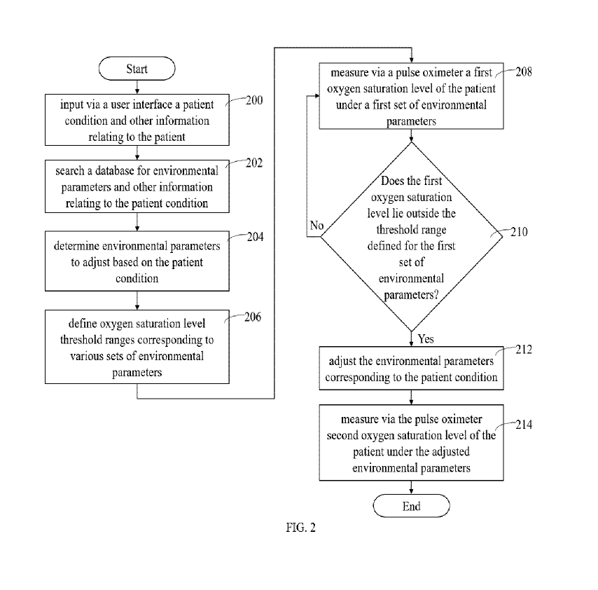

FIG. 2 illustrates a preferred method of the present invention. A user such as a medical practitioner inputs via the display with a user interface 102 the patient condition and other information relating to the patient (step 200). The inputted patient condition may include the disease and present status of the patient. For example, the medical practitioner inputs “chronic obstructive pulmonary disease (COPD)” in the field for the patient's disease in the user interface. Other information relating to the patient that may be inputted includes the patient's current medications, family history, and various patient information such as age, weight, and height. Next, the patient monitoring device 106 searches a database for environmental parameters and other information relating to the patient condition (step 202). Based on the information that the patient has a COPD, search results from the database show that humidity and temperature of a room and bed position affects the condition of the patient with COPD. Preferably, corresponding adjustment settings for the environmental parameters are also stored in the database. Afterwards, the patient monitoring device 106 determines environmental parameters to adjust based on the patient condition (step 204). The patient monitoring device 106 makes a priority decision that the environmental parameters that should be adjusted are humidity and bed position. Optionally, the priority decision is done manually by a medical professional.

Following the determination of the environmental parameters to adjust based on the patient condition (step 204), the patient monitoring device 106 defines oxygen saturation level threshold ranges corresponding to various sets of the environmental parameters (step 206). Examples of the defined oxygen saturation level threshold ranges (OSLTR) corresponding to various sets of environmental parameters are 69%-92% OSLTR for humidity and greater than 88% OSLTR for bed position. Preferably, the oxygen saturation level threshold ranges corresponding to various sets of the environmental parameters are based on the search results previously done by the patient monitoring device 106 (step 202). Alternatively, the oxygen saturation level threshold ranges corresponding to various sets of the environmental parameters are manually defined preferably by the medical practitioner via the display with the user interface 102.

After defining the oxygen saturation level threshold ranges (step 206), the pulse oximeter 100 measures a first oxygen saturation level of the patient under a first set of environmental parameters (step 208). Preferably, the first set of environmental parameters is the current environmental parameters of a room where the patient is staying. These environmental parameters can be the current settings for the devices that control these parameters. For example, the current humidity setting of a humidifier is 77% while the current bed position setting of an automatic bed is 30 degrees. For these environmental parameters, the measured oxygen saturation level of the patient is 86%. Subsequently, the patient monitoring device 106 determines if the first oxygen saturation level lies outside the threshold ranges defined for the first set of environmental parameters (step 210).

If the first oxygen saturation level does not lie outside the threshold ranges defined for the first set of environmental parameters, the pulse oximeter 100 continuously measures the oxygen saturation level of the patient under the first set of environmental parameters (step 208). Otherwise, the environment controller 104 adjusts the environmental parameters corresponding to the present patient condition (step 212). But if the measured oxygen saturation level in the previous example lies outside the threshold range for bed position, the patient monitoring device 106 then sends a control signal to the environment controller 104. The environment controller 104 then forwards the control signal to the automatic bed to adjust the angle of the bed from 30 degrees to 20 degrees. Finally, the pulse oximeter 100 measures a second oxygen saturation level of the patient under the adjusted environmental parameters (step 214). For example, the pulse oximeter 100 measures the oxygen saturation level of the patient lying on the bed that has just been adjusted to 20 degrees.

In another embodiment of the present invention, the determination of the environmental parameters to adjust (step 204), as described in FIG. 2, are also based on an outcome of a comparison between a measured oxygen saturation level and an oxygen saturation level threshold range.

In an exemplary embodiment of the present invention, a patient suffering from asthma is inside a hospital room at 90° F. and 78% humidity. Then, a medical practitioner inside the hospital room inputs in the user interface of the patient monitoring device 106 that the patient has an asthma. The patient monitoring device 106 searches a database for environmental parameters and other information relating to asthma. Search results show that temperature and humidity affects a patient suffering from an asthma. Also, the search results show that the optimum temperature and humidity for a person with asthma are 65° F.-75° F. and 35%-50%, respectively. Based on the search results, the patient monitoring device 106 determines that the temperature and the humidity of the room should be adjusted. The temperature and humidity are thus set with oxygen saturation level threshold ranges of 89%-92% and 88%-95%, respectively. Then, the pulse oximeter 100 measures the oxygen saturation level of the patient inside the room which is 87%. Subsequently, the patient monitoring device 106 determines that the measured oxygen saturation level lies outside the oxygen saturation level threshold ranges of temperature and humidity. Thereafter, the patient monitoring device 106 sends control signals to the environment controller 104. The environment controller 104 then sends an instruction to an HVAC unit to adjust the temperature to 70° F. and the humidity to 50%. Finally, the pulse oximeter 100 measures the oxygen saturation level of the patient again.

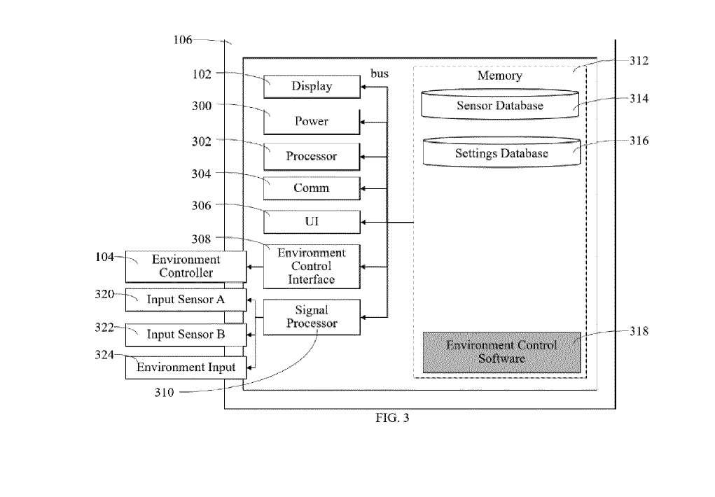

FIG. 3 illustrates another preferred embodiment of the patient monitoring device 106. The patient monitoring device 106 comprises a display 102, a power module 300, a processor 302, a communications module 304, a user interface 306, an environment control interface 308, a signal processor 310, and a memory 312. The memory 312 comprises a sensor database 314 and a settings database 316 and an environment control software 318 is stored in the memory 312. The signal processor 310 processes inputted data from the input sensor A 320, input sensor B 322, and environment input 324. The environment control interface 308 sends control signal to environment controller 104.

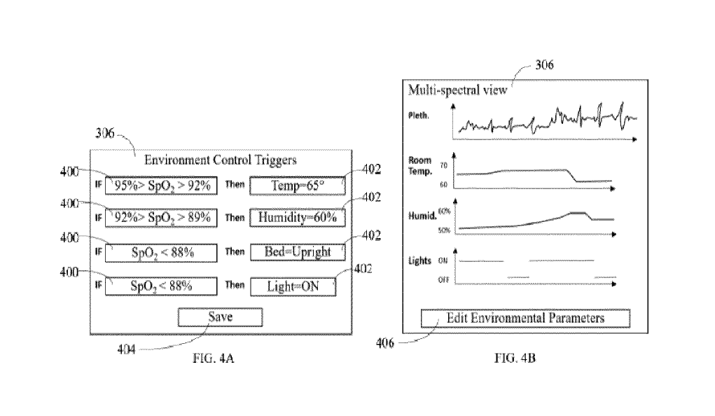

FIG. 4A illustrates an exemplary interface 306 for inputting environment control triggers in accordance with some embodiments. Condition fields 400 serve to allow user inputs for oxygen saturation level threshold ranges. On the other hand, response fields 402 serve to allow user inputs for the environmental parameters and any necessary environmental parameter value adjustments. The patient monitoring device 106 saves these inputs when button 404 is pressed by the user (e.g., medical practitioner). FIG. 4B illustrates an exemplary interface 306 for displaying plethysmograph waveform and environmental parameter data in accordance with some embodiments of the present invention. The environmental parameters data displayed on interface 306 are displayed in graphical form versus time. Pressing button 406 allows the user to edit environmental parameters.

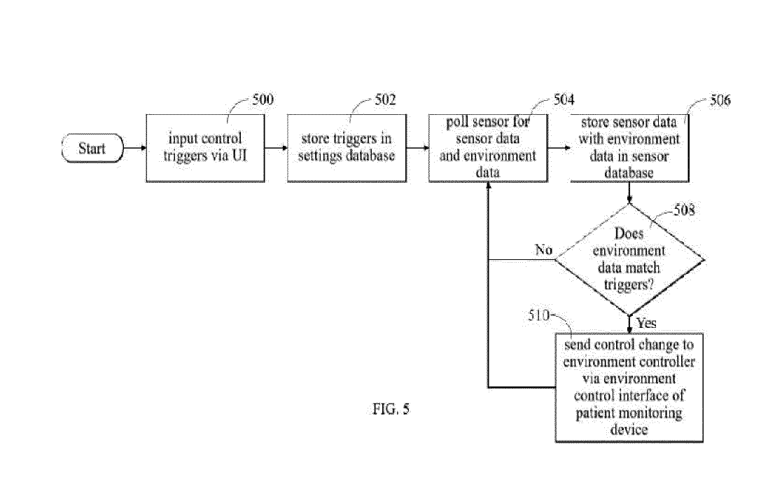

FIG. 5 illustrates the process of the environment control software 318 in accordance in some embodiment of the present invention. The user such as a medical practitioner inputs control triggers via user interface 306 described in FIG. 4A (step 500). The control triggers are the oxygen saturation level threshold ranges. Subsequently, the patient monitoring device 106 stores these triggers in the settings database 316 (step 502). After, the input sensor A 320, input sensor B 322, and environment input 324 are polled to check if sensor data and environment data are available (step 504). When sensor data and environment data are available, these data are stored by the patient monitoring device 106 in the sensor database 314 (step 506). Then, the patient monitoring device 106 checks if the environment data is consistent with the corresponding triggers (step 508). If the environment data does not match the corresponding triggers, the patient monitoring device 106 polls the input sensor A 320, input sensor B 322, and environment input 324 for sensor data and environment data (step 504). Alternatively, if the environment data match the corresponding triggers (falls within the oxygen saturation level threshold range), the patient monitoring device 106 sends control change signal to environment controller 104 via the environment control interface 308 (step 510). Finally, the patient monitoring device 106 polls again the input sensor A 320, input sensor B 322, and environment input 324 for sensor data and environment data (step 504).

As shown in FIG. 1, display 102 displays the plethysmograph waveform and SpO2 level collected by pulse oximeter 100. The temperature of the room is also displayed in the display 102. In another aspect of the invention, the patient monitoring device 106 and environment controller 104 are connected directly to each other via a physical link such as a USB connection.

The pulse oximeter 100 is preferably a portable pulse oximeter device worn on a patient's finger and adapted to measure the oxygen saturation level of the patient. The patient monitoring device 106 is preferably a stand-alone device connected to the display 102—which may be a laptop computer, a desktop computer, a mobile phone, a tablet computer, or a PDA. In another embodiment of the invention, the patient monitoring device 106 may reside within the display 102.

In accordance with the various embodiments of the present invention, the memory 312 may include high-speed random access memory or non-volatile memory such as magnetic disk storage devices, optical storage devices, or flash memory. Memory 312 may also store software instructions for facilitating processes, features and applications of the system disclosed in the invention. The communications module 304 may include any transmitter or receiver used for Wi-Fi, Bluetooth, infrared, NFC, radio frequency, cellular communication, visible light communication, Li-Fi, WiMax, ZigBee, fiber optic and other forms of wireless communication devices. Alternatively, the communications module 304 is a physical channel such as a USB cable or other wired forms of communication.

The present invention is not intended to be restricted to the several exemplary embodiments of the invention described above. Other variations that may be envisioned by those skilled in the art are intended to fall within the disclosure.

1. A system for adjusting environmental parameters for patient condition monitoring, the system comprising:
a pulse oximeter for measuring an oxygen saturation level of a patient;
a patient monitoring device connected to the pulse oximeter and a communications link, the patient monitoring device comprising:
a display device having a user interface for inputting one or more environment parameters;
a power module,
a processor,
a communications module,
an environment control interface,
a signal processor that processes data input from: one or more input sensors for acquiring sensor data, an environment input for acquiring an environment data, or both, and
a memory including a sensor database, a settings database, and an environment control software, and
an environment controller connected to the communications link, the environment controller adjusting the one or more environmental parameters;
wherein the user interface provides one or more condition fields that enable a user to input one or more oxygen saturation level threshold ranges, and one or more response fields that enable the user to input the environmental parameters or an adjustment to the environmental parameters.

2. The system of claim 1, wherein the environment controller sends one or more control signals to one or more devices that control the environment parameters.

3. The system of claim 2, wherein the one or more environmental parameters is selected from a temperature, a humidity, an air flow, an illumination, a bed position, and a combination thereof.

4. The system of claim 2, wherein the one or more devices that control the environmental parameters is selected from an HVAC unit, a humidifier, an LED bulb, an automatic bed, and a combination thereof.

5. The system of claim 2, wherein the one or more control signals are sent physically, wirelessly, or both.

6. (canceled)

7. (canceled)

8. The system of claim 1, wherein the user interface displays a plethysmograph waveform and the environmental parameters.

9. The system of claim 1, wherein execution of the environmental control software by the processor:
receives the one or more oxygen saturation level threshold ranges;
stores the one or more oxygen saturation level threshold ranges in the settings data base;
polls the input sensors and the environment input for availability of the sensor data and the environment data;
stores the sensor data and the environment data in the sensor database;
compares the stored environment data with the one or more oxygen saturation level threshold ranges; and
sends a control change signal to the environment controller when the stored environment data falls within the one or more oxygen saturation level threshold ranges.

10. A method for adjusting environmental parameters for patient condition monitoring, the method comprising:
inputting, via one or more user inputs on a user interface, a patient condition and other information relating to the patient;
searching a database for environmental parameters and other information relating to the patient condition;
determining environmental parameters to adjust based on the patient condition;
defining oxygen saturation level threshold ranges corresponding to various sets of environmental parameters;
measuring via a pulse oximeter a first oxygen saturation level of the patient under a first set of environmental parameters;
adjusting the environmental parameters corresponding to the patient condition if the first oxygen saturation level lies outside the threshold ranges defined for the first set of environmental parameters; and
measuring a second oxygen saturation level of the patient under the adjusted environmental parameters.
